(19) United States
(12) Patent Application Publication
(10) Pub. No.: US 2020/0279629 A1
(43) Pub. Date: Sep. 3, 2020

(54) SYSTEM AND METHOD FOR GATHERING AND ANALYZING HUMAN EXPERIENCE REPORTS

(71) Applicant: MEDICINE DIFFERENTIATION ANALYTICS, LLC., Mendham, NJ (US)

(72) Inventors: Eileen MORRISSEY, Mendham, NJ (US); Jim ANDERSON, Scottdale, AZ (US)

(73) Assignee: MEDICINE DIFFERENTIATION ANALYTICS, LLC., MENDHAM, NJ (US)

(21) Appl. No.: 16/804,445

(22) Filed: Feb. 28, 2020

Related U.S. Application Data
(60) Provisional application No. 62/812,570, filed on Mar. 1, 2019.

(51) Int. Cl. G16H 20/10; G16H 70/40; G16H 10/60; G16H 50/70; G06Q 30/02

(52) U.S. Cl. CPC G16H 20/10; G16H 70/40; G06Q 30/0201; G16H 50/70; G16H 10/60

(57) ABSTRACT

A system and method for gathering and analyzing electronic records. The method comprises collecting user value statements, determining personal product differentiation (PPD) scores based on the user value statements, collecting experiences related to a user reaction to a product, analyzing the collected experiences based on the PPDs, and generating at least a recommendation of a preferred product for the user.

### CROSS REFERENCE TO RELATED APPLICATIONS

This application claims the benefit of U.S. provisional application 62/812,570 filed on Mar. 1, 2019, the contents of which are herein incorporated by reference.

### TECHNICAL FIELD

The present disclosure relates generally to systems and methods for gathering and analyzing human experience reports, and, specifically, to systems and methods for determining product differentiation given a unique set of requirements.

### BACKGROUND

Developments in the pharmaceutical and healthcare industries, over the past twenty years, have allowed for the creation and subsequent application of a variety of new drugs and related treatments. As new drugs are prescribed and the success of treatment is evaluated, side effects, negative drug interactions, and contra-indications may be observed. Potential negative effects resulting from the use of new drugs can include exacerbation of the treated condition, the creation of complications, and serious concerns including, at the most extreme, death. Negative drug effects may be costly to patients in the aggregate and may not be discovered until after a drug is available on the market.

Management of treatment contra-indications and negative effects may be time-consuming, difficult, or even impossible, given the variability of success that a given drug may have in treating a condition in a specific patient. Furthermore, the information provided by drug manufacturers may be under-informative, such as content-limited TV and print ads, or over-informative, as may be the case for complex detail labels, which may be dozens of pages long and which may contain technical phraseology which consumers may not comprehend.

In addition to physicians and patients, governments and insurance companies may be invested in the management of patients' treatments. The addition of the payors' interests into the drug-selection calculation adds variables beyond the efficacy and safety of a treatment, factors which may be most relevant to physicians and patients. As governments and insurers may be required to shoulder the burden of patients' treatments, depending on the specific arrangements, factors including price and duration of treatment, among others, may factor into selection of a treatment method. The factors relevant to a payor's decision to pay for a treatment may prohibit the selection of certain medications, even after the patient and doctor have selected a course of treatment. These rejections, as well as the potential for possible negative interactions from payors' suggested substitute treatments, may cause unnecessary delays in treatment and may cause additional drug interactions. These difficulties may be mitigated or avoided by considering drug interactions, treatment preferences, and healthcare formularies at the time that a treatment decision is made.

As a result of the factors considered by the various interested parties, patients and physicians are often left to optimize treatment by selecting a drug which best suits the needs of the patient and the payor. Factors which may weigh on the drug-selection analysis include, without limitation, whether the drug addresses the patient's symptoms, whether the drug is included in a patient's healthcare formulary, i.e., whether a payor will pay for the treatment, whether the method of administration suits the patient's preference, whether the drug's side effects conflict with the patient's prioritized list of undesirable side effects, and whether the treatment will negatively interact with any of the patient's other drugs, over the counter medications (OTCs), or nutritionals.

In addition to a solution to the difficulties of selecting an appropriate treatment which serves the needs of patients, physicians, and payors, monitoring of a patient's response to the treatment may be equally valuable. As patients may consume multiple drugs, OTCs, and nutritionals, the potential for negative interactions may increase. In order to support on-going treatment of a specific patient, and to collect data regarding drug interactions, data from patient reports, physician analyses, and wearable device records may improve the quality of care for a given patient, as well as for those prescribed the same treatments.

In addition to improving treatment outcomes, the collection of drug interaction data may benefit medication manufacturers by supporting the companies' regulatory obligations to provide ongoing real-world treatment and evaluation data regarding rates of success in patients and reported negative interactions and effects. To address a lack of post-approval data, particularly concerning drug interactions and ongoing patient monitoring, the Food and Drug Administration has emphasized the need for collection and preservation of Real-World Data (RWD) and Real-World Evidence (RWE). RWD and RWE are useful to the FDA for potential applications in monitoring postmarket safety and adverse events and to make regulatory decisions, to the healthcare community for the development of guidelines and decision support tools for clinical practice, and to medical product developers for application in clinical trial design and in generation of new treatment approaches. Although analysis of RWE may allow for improvements to treatment, diagnosis, and other aspects of medical practice, collection of RWE is stymied by challenges including collaboration barriers, a lack of infrastructure, inconsistent methodologies, privacy concerns, and lack of standardization. The need for effective RWE collection, categorization, and archival solutions remains unmet, despite a growing maket for RWE products and solutions, a market which is expected to grow as large as $1.64 billion by 2024. Due to the large volume of relevant data potentially available, collecting, recording, and correlating the relevant data may be a task too onerous for any one physician or manufacturer.

The scope challenges presented, regarding selection and monitoring, may be appreciated by consideration of several relevant key figures. Highlighting the value of effective monitoring and selection methods and systems, over one million adverse drug reactions (ADRs) are reported annually, with over 100,000 deaths arising therefrom, ADRs which are estimated to cost the U.S. up to $30 billion annually. Furthermore, between 44,000 and 98,000 deaths may result each year from medical error. The difficulty of addressing these challenges owes largely to the scale of the issue, with more than half of the U.S. regularly taking an average of four prescription medications and older adults often taking more than fifteen, in addition to over-the-counter (OTC) medications, vitamins, and dietary supplements. This issue of scale is further compounded by projected increases in diagnoses, with some estimates projecting that more than 32 million Americans over sixty-five will have two or more chronic diseases by 2030, and by the complexity of diagnosis, treatment, and evaluation. As an example of the complexity of prescription and ADR management, a single multiple sclerosis drug is known to have at least 400 major interactions with other medications, reducing the likelihood of comprehensive evaluation and selection of complementary medications, particularly across many complex disease categories and across millions of patients.

Further, the question of cost, relevant to both patients and payors, may be addressed by the application of evolving technology. The high cost of treatment derives, in part, from the costs of advertising the treatments to physicians and potential patients. As drug companies may spend large amounts advertising a treatment broadly, taking out print and TV ads in the hope of reaching the subset of patients who would respond favorably to a given medication, the costs of this advertising may be reflected in the costs of the treatment. By selectively targeting certain physicians or patients, medication manufacturers may reduce their costs, a reduction which may be reflected in medication prices. However, targeted advertisement is limited by factors including the broad reach of print and TV ads, the difficulty of selecting relevant potential patients and physicians, and the difficulty of targeting advertisements to those parties who may be interested.

It would, therefore, be advantageous to provide an efficient and effective evaluation solution for determining the best treatment plan for patients.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for gathering and analyzing electronic records. The method comprises the steps of collecting user value statements, determining personal product differentiation (PPD) scores based on the user value statements; collecting experiences related to a user reaction to a product; analyzing the collected experiences based on the PPDs; and generating at least a recommendation of a preferred product for the user.

Certain embodiments disclosed herein also include a non-transitory computer readable medium having stored thereon instructions for causing a processing circuitry to execute a process. The process comprises the steps of collecting user value statements; determining personal product differentiation (PPD) scores based on the user value statements; collecting experiences related to a user reaction to a product; analyzing the collected experiences based on the PPDs; and generating at least a recommendation of a preferred product for the user.

Certain embodiments disclosed herein also include a system for for gathering and analyzing electronic records. The system comprises a processing circuitry; and a memory, the memory containing instructions that, when executed by the processing circuitry, configure the system to collect user value statements; determine personal product differentiation (PPD) scores based on the user value statements; collect experiences related to a user reaction to a product; analyze the collected experiences based on the PPDs; and generate at least a recommendation of a preferred product for the user.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a flowchart depicting a method for completing the patient product differentiation analysis, monitoring effectiveness of the treatment selected, performing AI analysis on the resulting treatment experiences, and providing targeted marketing, according to an embodiment.

FIG. 2 is a flowchart depicting a method for developing patient value statements, according to an embodiment.

FIG. 3 is a flowchart depicting a method for gathering human experience reports, according to an embodiment.

FIG. 4 is a flowchart depicting a method for gathering the patient's healthcare formulary regarding treatment options in a disease area, according to an embodiment.

FIG. 5 is a flowchart depicting a method for gathering drug interactions, according to an embodiment.

FIG. 6 is an illustration depicting an application of gathered drug interactions, according to an embodiment.

FIG. 7 is a flowchart depicting a method for gathering product details, according to an embodiment.

FIG. 8 is a flowchart depicting a method for determining personal product differentiation (PPD) and reporting results, according to an embodiment.

FIG. 9 is a flowchart depicting a method for continually monitoring a selected product's efficacy and safety, given the unique monitoring needs of the specific product and the patient's medical history, according to an embodiment.

FIG. 10 is a flowchart depicting a method for connecting two databases, including a human experience report database, and performing artificial intelligence and machine learning analyses, according to an embodiment.

FIG. 11 is a flowchart depicting a method for providing targeted marketing data, according to an embodiment.

FIG. 12 is a block diagram illustrating how the three proprietary databases interact, according to an embodiment.

FIG. 13 is a block diagram illustrating a system utilized to execute the processes described herein, according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be plural, and vice versa, with no loss of generality. In the drawings, like numerals refer to like parts through several views.

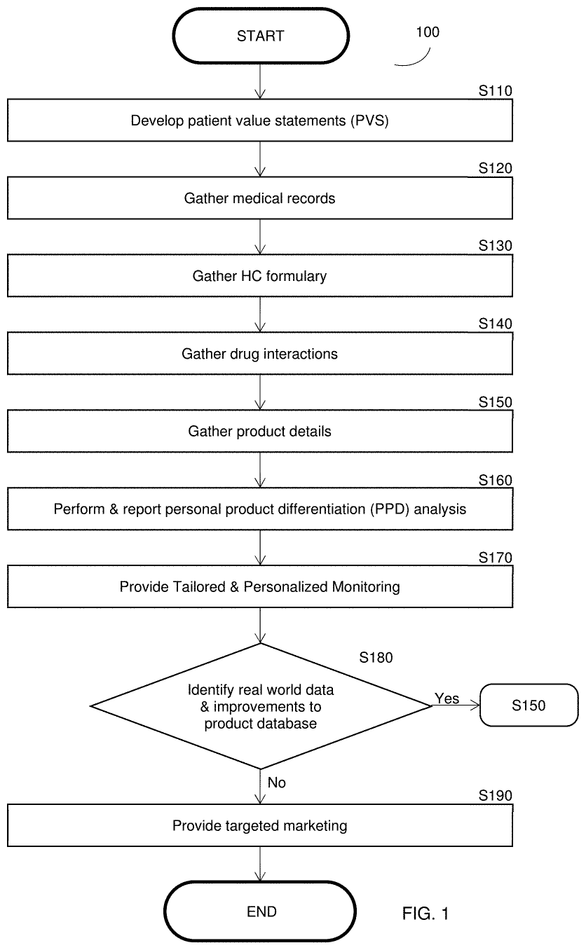

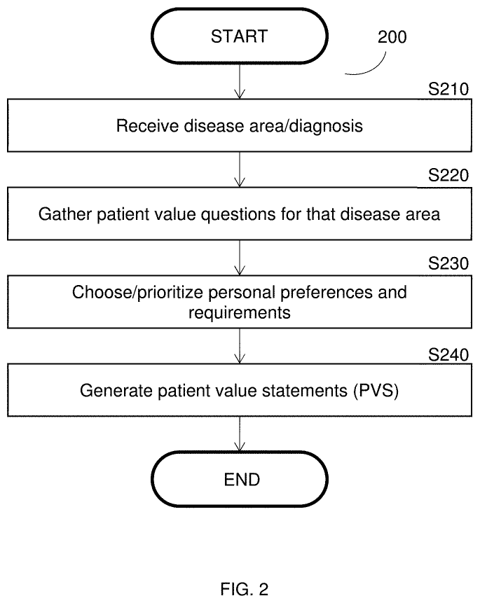

FIG. 1 is an example flowchart depicting a method for completing the patient product differentiation analysis, monitoring effectiveness of the treatment selected, performing AI analysis on the resulting treatment experiences, and providing targeted marketing, according to an embodiment. At S110, a patient value statement (PVS) is developed. A method of developing a patient value statement, according to an embodiment, is illustrated in FIG. 2 below.

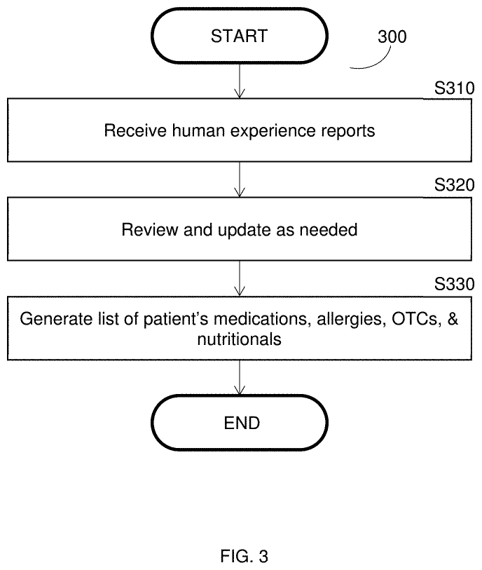

At S120, medical records are gathered. A method for gathering medical records, according to an embodiment, is illustrated in FIG. 3 below.

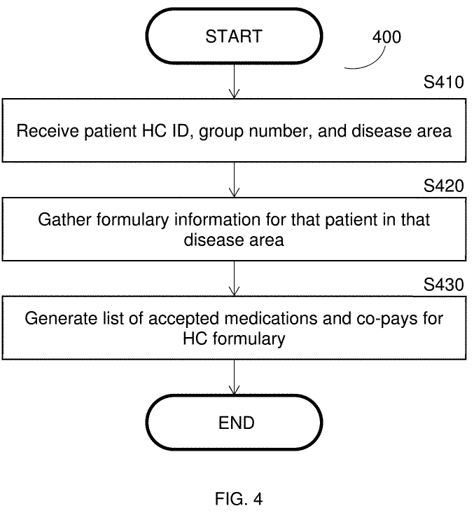

At S130, healthcare formulary is gathered. A method for gathering healthcare formulary, according to an embodiment, is illustrated in FIG. 4 below.

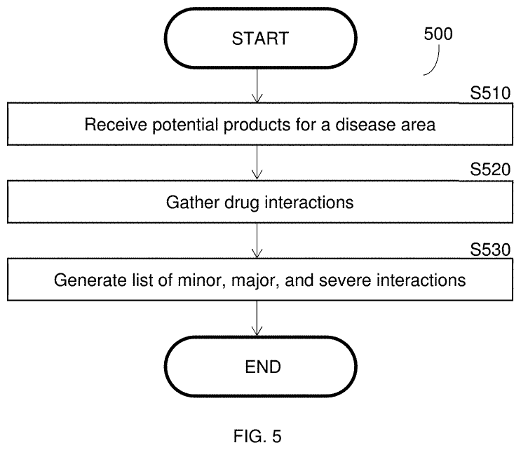

At S140, drug interactions are gathered. A method for gathering drug interactions, according to an embodiment, is illustrated in FIG. 5 below.

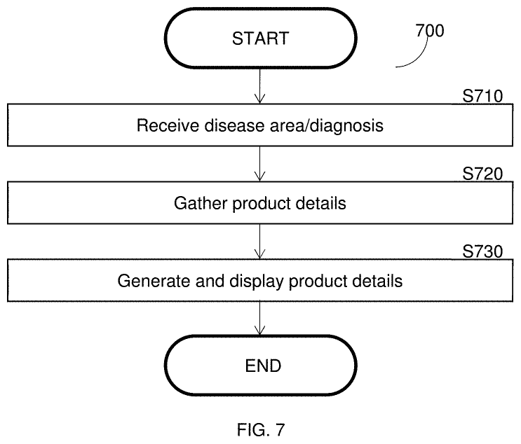

At S150, product details are gathered. A method for gathering product details, according to an embodiment, is illustrated in FIG. 7 below.

It should be noted that steps S120 through S150 may be performed in any order, including simultaneously, without deviating from the scope of the disclosure. These processes combine inputs with unique processing steps, rendering outputs independent of one another.

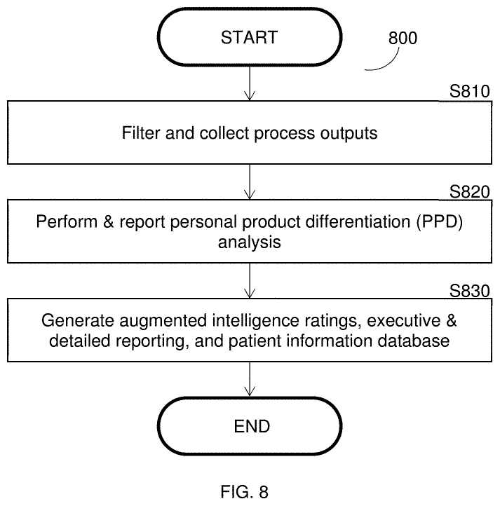

At S160, personal product differentiation (PPD) analysis is performed and reported. A method for performing and reporting PPD analyses, according to an embodiment, is illustrated in FIG. 8 below.

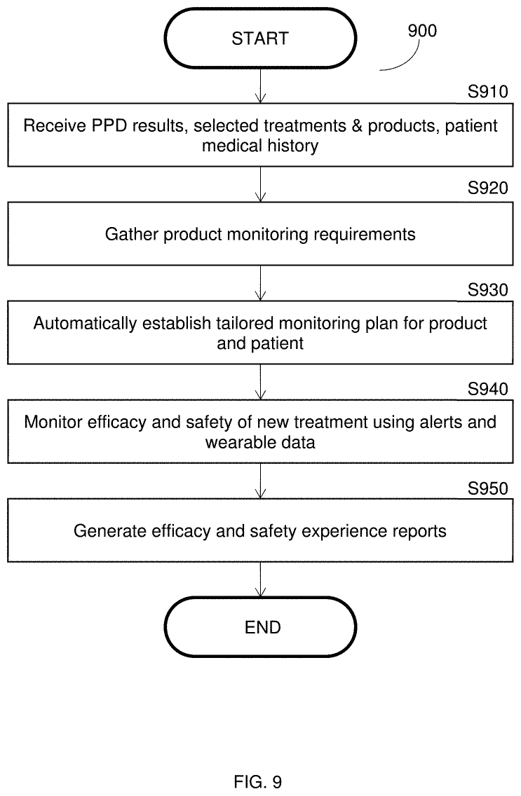

At S170, tailored and personalized monitoring is provided. A method for providing tailored and personalized monitoring is illustrated, according to an embodiment, in FIG. 9 below.

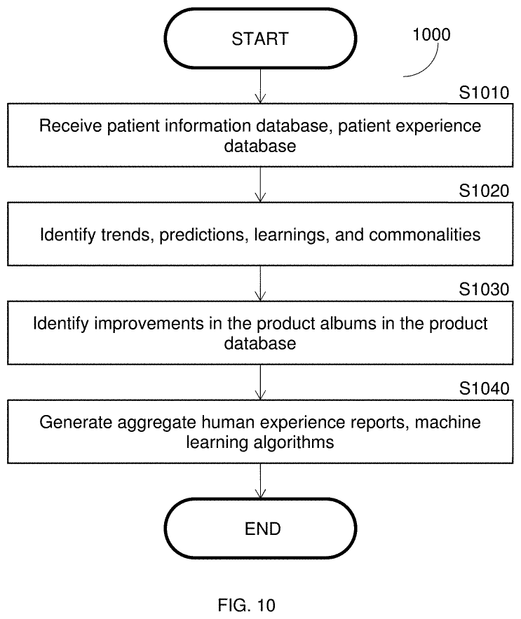

At S180, real world data and improvements to the product database are identified. A method for identifying real world data and improvements to the product database, according to an embodiment, is illustrated in FIG. 10 below. In an embodiment, where real world data and improvements to the product database remain to be identified, the method according to FIG. 1 reverts to step S150, the gathering of product details. In an embodiment, where no real world data and improvements to the product database remain to be identified, the method according to FIG. 1 proceeds to subsequent steps.

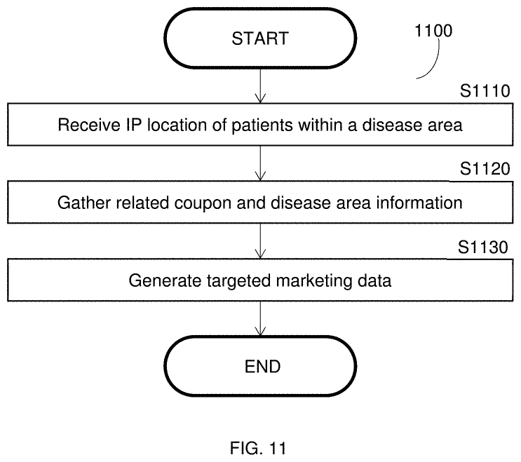

At S190, targeted marketing is provided. A method for providing targeted marketing, according to an embodiment, is illustrated in FIG. 11 below.

It should be noted that S190 may occur in a different order than that shown in the diagram 100. That is, step S190 may occur prior to S170, after S180, or concurrently with the execution of either step S170 and S180 without any deviation from the scope of the disclosure. In an example embodiment S190 occurs after S180 where no real world data and improvements to the product database remain to be identified at S180.

FIG. 2 is an example flowchart 200 depicting a method for developing patient value statements, according to an embodiment. At S210, a disease area or diagnosis is received. The received disease area may be a field of medical practice such as, as examples and without limitation, pulmonary, cardiac, skeletal, neurological, and other, like, disease areas. The received diagnosis may be a diagnosis of a specific condition such as, as examples and without limitation, asthma, lupus, COPD, and other, like, diagnoses. The disease area or diagnosis may be determined by a healthcare provider, such as a physician.

At S220, patient value questions for the disease area or diagnosis, received at S210, are gathered. Patient value questions may be questions selected for patients with conditions falling within a certain disease area or diagnosis and may prompt the patient to prioritize the patient's preferences and define the patient's medical situation. Patient value questions may include, as examples and without limitation, “which specific symptoms are most problematic for the patient,” “which side effects does the patient want least or not at all,” “which administration method does the patient prefer (oral, injected, doctor visits required, extra blood tests, etc.),” and other, like, questions.

At S230, personal preferences and requirements are chosen and prioritized. Personal preferences and requirements may be chosen and prioritized based on the patient's answers to the patient value questions gathered at S220. At S230, the patient value questions gathered at S220 may be presented to the patient for response, in order to choose or prioritize personal preferences and requirements. Patients may choose or prioritize personal preferences and requirements by answering patient value questions on a personal electronic device, by responding to a physician's questions, by accessing a health kiosk in a doctor's office or other public place, or by other similar means.

At S240, patient value statements are generated. The generated patient value statements may describe a patient's unique medical situation. The patient value statements may be collected, archived, stored, or otherwise preserved for subsequent application in determination of a personal product differentiation.

FIG. 3 is an example flowchart 300 depicting a method for gathering human experience reports, according to an embodiment. At S310 human experience reports are received. In an embodiment, the received human experience reports may be patient medical records. In an embodiment, receiving human experience reports may include accessing electronic medical records. In addition, receiving human experience reports may also include processing human experience reports manually uploaded by a user. The human experience reports uploaded by a user may be in the form of printed-and-scanned reports, reports entered in plain text or other text formats, reports uploaded via a web application, and other, like, report uploads

At S320, the human experience reports are reviewed and uploaded as needed. In an embodiment, a patient, a healthcare provider, or both, may be prompted to review the human experience reports and to update as needed.

At S330, a list of the patient's medications, allergies, OTCs, and nutritionals is generated. The list of medications, allergies, OTCs, and nutritionals may be generated by analysis of the medical records received at S310 using methods including, without limitation, keyword matching, field-value extraction, and other like techniques. In an embodiment, the list of medications, allergies, OTCs, and nutritionals may be used subsequently to analyze potential negative drug interactions during the determination of a personal product differentiation.

FIG. 4 is an example flowchart 400 depicting a method for gathering the patient's healthcare formulary regarding treatment options in a disease area, according to an embodiment. At S410, a patient's healthcare ID, group number, and disease area are received.

At S420, formulary information for a patient in that disease area is gathered. Based on the information received at S410, formulary may be determined based on the patient's condition and healthcare status. Changes in healthcare formulary may include additions to a list of medications pre-approved by an insurance company, variations in a medication pricing schedule, and other, like, formulary-related changes. Formulary information may be gathered by means including, without limitation, receiving updated formulary information from health insurance companies, governments or other payors, requesting updated formulary information from health insurance companies, governments, or other payors, automatically searching webpages for relevant information, a process which may be known as “crawling,” and other, like, means.

At S430, a list of accepted medications and co-pays for the healthcare (HC) formulary is generated. Based on the formulary gathered at S420 and the disease area received at S410, a list of medications for a given condition may be generated. In an embodiment, the list may include, without limitations, medication names, brand names, generic equivalents, dosage information, known side effects, costs without insurance, co-pays, and other, like, information. Furthermore, in an embodiment, the list may include medications not covered under the patient's healthcare formulary, medications covered under the patient's HC formulary, or a combination of the two.

FIG. 5 is an example flowchart 500 depicting a method for gathering drug interactions, according to an embodiment. At S510, potential products are received for a disease area. Products for a disease area may be identified using techniques including, without limitation, keyword searching, AI-driven selection, and the like. Potential products for a disease area may be received from a product database or other database repository, or from another store of product information.

At S520, drug interactions are gathered. Drug interactions may be identified automatically based on data regarding a particular drug. Drug interaction analysis may include the analysis of a patient's personal information including, without limitation, disease area, diagnosis, other drugs, allergies, OTCs, and nutritionals, and other, like, information. The drug interaction gathering at S520 may be performed proactively, allowing a patient to understand the possible interactions before starting a course of medication.

At S530, a list of minor, major, and severe interactions is generated. The list of minor, major, and severe interactions may include minor interactions, major interactions, severe interactions, and combinations thereof. The list of interactions may be output as a readout on a physician or patient's computer, mobile device, or other device, as print media sent by mail, as email, text message, or digital message of another kind, or as a feature included in an accessible webpage

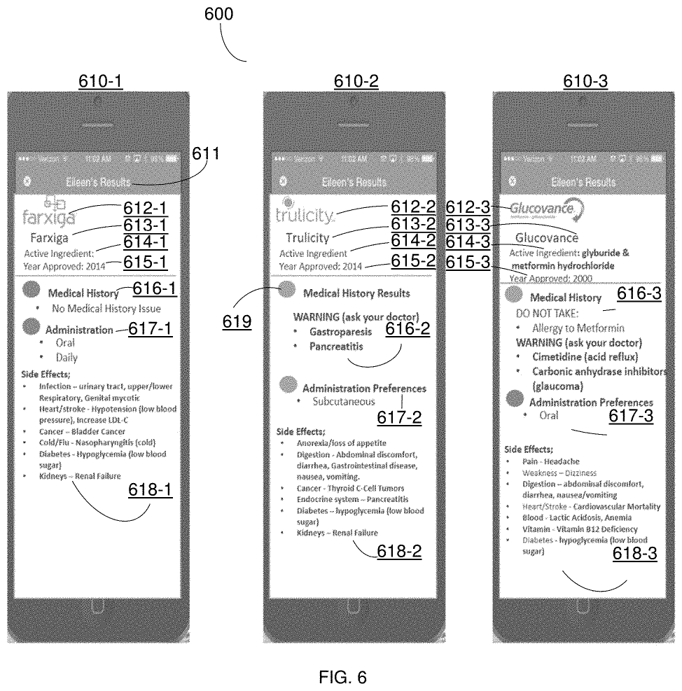

FIG. 6 is an illustration 600 depicting an application of gathered drug interactions, according to an embodiment. In an embodiment, the list of minor, major, and severe interactions generated at S530 may be displayed through a user interface, 610-1 through 610-3, on a smartphone, computer, or other, like, device. The user interface may include a description 611 identifying a particular user and indicating that the displayed information is personalized to that particular user.

The user interface 610 may display general information regarding a particular drug, therapeutic, or other treatment including, without limitation, a product logo, 612-1 through 612-3, a product name, 613-1 through 613-3, a listing of active ingredients, 614-1 through 614-3, an approval year, 615-1 through 615-3, and other, like, information. The general information displayed regarding a particular drug, therapeutic, or other treatment, may be displayed to allow a patient, physician, healthcare payor, or other party to identify a particular drug, therapeutic, or other treatment by logo, brand name, or active ingredient, and to assess additional relevant information including, without limitation, the period which has elapsed since approval.

Further, the user interface 610 may display personalized information regarding a particular drug, therapeutic, or other treatment including, without limitation, medical history issues and warnings, 616-1 through 616-3, administration routes, methods, preferences, and other, like, administration information, 617-1 through 617-3, side effects, 618-1 through 618-3, and other, like, personalized information. The displayed medical history issues and warnings, 616-1 through 616-3, may include allergy information, condition contra-indications, and other, like, information, drawn from sources including, without limitation, analysis of patient medical records, drug labels and associated information, experience reports, and other, like, sources. The displayed administration routes, methods, preferences, and other, like, administration information, 617-1 through 617-3, may include, without limitation, administration routes, dosages, schedules, patient preferences, and other, like, administration information, drawn from sources including, without limitation, patient preference inputs, drug labels and the like, other, like, sources of information, and any combination thereof. The displayed side effects, 618-1 through 618-3, may include side effects drawn from sources including, without limitation, drug labels and associated sources of information, patient experience reports, other, like, sources, and any combination thereof.

In an embodiment, the user interface 610 may include displayed indicator labels 619 associated with the displayed medical history issues and warnings, 616-1 through 616-3, and the displayed administration routes, methods, preferences, and other, like, administration information, 617-1 through 617-3. The displayed indicator labels 619 may be colored to indicate the severity of a detected issue, interaction, or contra-indication. In an embodiment, a green indicator label 619 may be applied where no issue is detected, a yellow indicator label 619 may be applied where a minor issue is detected, and a red indicator label 619 may be applied where a major or severe issue is detected, alerting patients, physicians, pharmacists, healthcare payors, and other parties, of medication or other treatment issues which may arise from conflicts between, as examples and without limitation, a patient's medication and existing conditions, between the patient's other medications, or between the patient's preferences and the labeled or reported administration methods or other interactions. The display of indicator labels 619, and the respective types of the indicator labels 619 displayed, may arise from analyses of data including, without limitation, patient medical records, patient preferences, drug labels, patient experience reports, and other, like, datasets.

FIG. 7 is an example flowchart 700 depicting a method for gathering product details, according to an embodiment. At S710, a disease area identifier, diagnosis, or both, is received. The received disease area or diagnosis may be a disease area or diagnosis previously generated for a patient, or may be provided to the system as an input entered in isolation for such purposes as, without limitation, researching products. The received disease area or diagnosis may be identified by a physician with respect to a specific patient, or may be generated, without application to a specific patient, in order to research product details.

At S720, product details are gathered. The gathered product details may include information from sources including, without limitation, medication labels, websites, brochures, advertisements, medical texts, and other like sources of information. In an embodiment, the received product details may be received from a proprietary product database. The proprietary product database may include information categories including, without limitation, uses, side effects, interactions, administration methods, and other like categories. The proprietary database may be continually updated to include new information regarding changes in labels or new product labels. In an embodiment, the product details gathered at S720 may be included in a subsequent determination of a personal product differentiation.

At S730, relevant product details, from the product details gathered at S720, are generated and displayed. The details may be reviewed by physicians, patients, and other interested parties. Such product details may include, without limitation, product uses, side effects, administration methods, interactions and severity, contraindications and severity, warnings and severity, adverse reactions and severity, and other, like, details. In an embodiment, relevant generated product details may be displayed via methods including, without limitation, display on a computer, mobile, or other device, display on a printed readout generated by configured compatible hardware, an email, text, or other notification, and other, like, methods.

FIG. 8 is an example flowchart 800 depicting a method for determining personal product differentiation (PPD) and reporting results, according to an embodiment. At S810, process outputs are filtered and collected. The process outputs filtered and collected may be filtered by grouping datasets, machine learning (ML) insights, and other, like, features, according to one or more unique or distinguishing data features, tags, labels, or other, like, identifiers. In an embodiment, the process outputs filtered and collected at S810 may include the outputs of prior operations. Specifically, according to an embodiment, the process outputs collected at S810 may include patient value statements, lists of a patient's medications, allergies, OTCs, and nutritionals, lists of accepted medications and co-pays for the patient's formularies, lists of major, minor, and severe interactions, product details, and any combination thereof.

At S820, a personal product differentiation analysis is performed and reported. In an embodiment, the analysis may include determination of a best-fit product, considering a patient's unique details. The patient and physician may review the reported analysis by viewing an organized list of products which may be relevant to the patient's disease area or diagnosis, where each product listed may be assigned a ranking score during analysis. Additionally, the analysis and reporting performed at S820 may include an interactive prioritization, whereby patients and physicians may be allowed to work together to dynamically assign weights to factors which are important to the patient, altering the analysis of specific products. The analysis and reporting performed at S820 may be further configured to allow patients and physicians to segment lists of products, such that only products indicated for, for example, a certain route of administration or lack of a certain side effect, are displayed.

At S830, augmented intelligence ratings, executive and detailed reporting, and patient information database entries are generated. The augmented intelligence ratings may include product scoring information and recommendations based on a patient's unique information, details regarding the medications available, and the application of artificial intelligence, machine learning algorithms, or both. The executive and detailed reporting may include information reported at S820, reports generated by artificial intelligence, machine learning, or both, brief summaries distilled from larger bodies of information, detailed summaries generated from all available information, and other, like, reports. The generation of patient information database entries may include the addition of entries to the patient information database or databases. The patient information database or databases may include, without limitation, blinded information on a patient's demographics, medications, preferences, region, other, like, information, and any combination thereof.

FIG. 9 is a flowchart 900 depicting a method for continually monitoring a selected product's efficacy and safety, given the unique monitoring needs of the specific product and the patient's medical history, according to an embodiment. At S910, personal product differentiation (PPD results), selected treatments and products, and patient medical history are received. The PPD results may include results generated by a process similar or identical to those processes described above with respect to figures one through five and figure seven, results generated independent of a patient and used for research, and the like. The selected treatments and products may include ordered and unordered lists of treatments and products, lists of treatments and products generated automatically, lists of treatments and products generated with human supervision, and any combination thereof. The received patient medical history may include information collected at an earlier stage, information entered for the purpose of continually monitoring a selected product's efficacy and safety, unverified information collected by inference or hypotheses, other, like, information, and any combination thereof.

At S920, product monitoring requirements are gathered. The gathered product monitoring requirements may include monitoring requirements for individual products. The monitoring requirements gathered at S920 may be received from a product database or other, like, store of product data. Furthermore, monitoring requirements may be entered manually by interested parties including, without limitation, patients, physicians, healthcare payors, medication manufacturers, and other, like, interested parties.

At S930, tailored product and patient monitoring plans are automatically established. In an embodiment, the automatically established tailored monitoring plan for a product or patient may include information received at S910, combined with information gathered at S920. Further, in an embodiment, the automatically established tailored monitoring plan may include subsets of the information received at S910, subsets of the information gathered at S920, or a subset of the combination of the two.

At S940, the efficacy and safety of a new treatment is monitored using alerts and wearable data. In an embodiment, alerts may be standard by disease area and may be prioritized, timed, and tailored to a specific medication, a specific patient, or a combination of the two. Furthermore, in an embodiment, data may be received from wearable devices to further monitor the health of the patient in response to a new treatment.

At S950, efficacy and safety experience reports are generated. The safety and efficacy experience reports of S950 may include health feedback and treatment responses. The feedback and response data included at S950 may be prioritized and sent to a healthcare provider or physician for review and further action, as needed. In an embodiment, the feedback and treatment response outputs may be updated continually. In an embodiment, feedback and treatment responses may be saved to a proprietary patient experience database, another database, or another repository, server, other data storage, or any combination thereof. In an embodiment, feedback and treatment responses, and other data saved to a patient experience database, as well as other data containing information identifying a patient, either directly or indirectly, may be anonymized, obfuscated, or otherwise de-identified.

FIG. 10 is a flowchart 1000 depicting a method for connecting two databases, including a human experience report database, and performing artificial intelligence and machine learning analyses, according to an embodiment. At S1010, according to an embodiment, database connections to a patient information database and patient experience database are received. The connections to the patient information database and patient experience database may be received in any order, including concurrently.

At S1020, trends, predictions, learnings, and commonalities are identified. The trends, predictions, learnings, and commonalities identified may be drawn from one database, both databases, or any combination of various portions of each database, separately and together. In an embodiment, the identification of trends, predictions, learnings, and commonalities may be effected by the use of artificial intelligence (AI) methods, machine learning (ML) methods, such as, as examples and without limitation, supervised methods, unsupervised methods, other, like, ML methods, and any combination thereof, or a combination of the two. In such an embodiment, the identification of trends and predictions using AI, ML, or a combination of the two, may yield trends and predictions applicable as real-world data (RWD). In an example embodiment, AI methods may be applied to standard categories including, as examples and without limitation, disease areas, medications, side effects, and the like. Furthermore, in an example embodiment, the RWD generated may include indications of efficacy of a certain drug for patients with a specific condition, statistical risk of a specific condition in patients of a given demographic, or influence of medications, allergies, OTCs, or nutritionals on disease. In an embodiment, the trends, predictions, learnings, and commonalities identified at S1020 may be reported to organizations in fields including, without limitation, research, marketing, regulation, healthcare, and the like.

At S1030, improvements in the product albums in the product database are identified. In an embodiment, the trends, predictions, learnings, and commonalities identified at S1020 may be applied to identify improvements to the product database. The identified improvements made to the product database may include, without limitation, improvements to treatment selection algorithms, newly-generated or newly-identified patient or treatment data such as, for example, the presence of new side effects in a certain population, other, like improvements, and any combination thereof.

At S1040, aggregated human experience reports and machine learning analytics are generated. In an embodiment, the generated aggregated human experience reports and machine learning analytics may include updating product database entries with the improvements identified at S1030. Additionally, the generated aggregated human experience reports and machine learning analytics may include trends, predictions, learnings, and commonalities identified at S1020, and may also include the patient information and patient experiences databases received at S1010, reconsiderations of previously-identified trends in light of newly-generated algorithms, other like data, and any combination of those outputs considered, including statistics and data presented in comparison with average and target data.

FIG. 11 is a flowchart 1100 depicting a method for providing targeted marketing data, according to an embodiment. At S1110, the IP location of patients for a given disease area is received. The received patient information, including the received IP location, connection data, and other, like, patient information, may be blinded, obfuscated, or otherwise anonymized to remove information identifying individual patients.

At S1120, related coupon and disease area information is gathered. The gathered related coupon and disease area information may include manufacturer's advertising campaigns, manufacturer and store coupons for certain products, information regarding patients having a certain diagnosis or within a certain disease area, other like information, and combinations thereof.

At S1130, targeted marketing data is generated. The generated targeted marketing data may include advertisements, coupons, alerts, notifications, other like features, and combinations thereof. In an embodiment, targeted marketing data may be addressed to patients and physicians as notifications on a user device or computer, emails, texts, or other messages, print media sent by mail, or by other, like means.

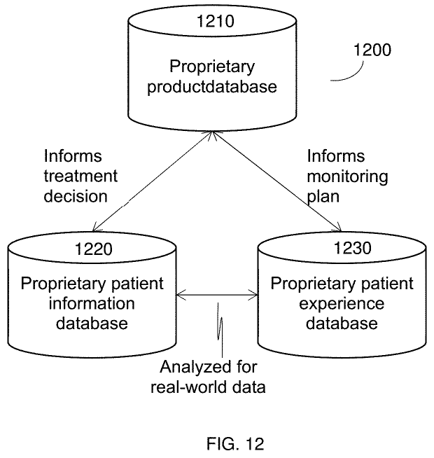

FIG. 12 is an example block diagram 1200 illustrating how the three proprietary databases interact, according to an embodiment. In the example embodiment, each of the three databases, the proprietary product database 1210, the proprietary patient information database 1220, and the proprietary patient experience database 1230, inform each other to allow for personalized treatment decisions, building a tailored remote monitoring plan, and identifying real world data. In the example embodiment, the proprietary product database 1210 and the proprietary patient information database 1220 may interact in the process of informing treatment decisions. In the example embodiment, the proprietary patient information database 1220 and the proprietary patient experience database 1230 may interact in the analysis of real-world data. In the example embodiment, the proprietary product database and the proprietary patient experience database 1230 may interact in the process of informing a monitoring plan.

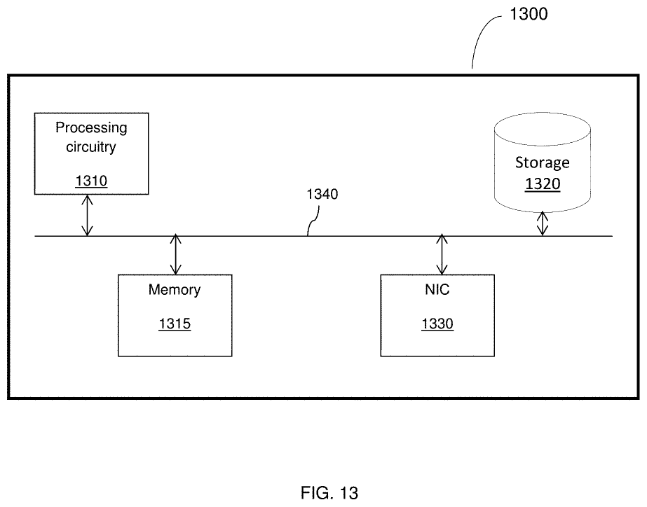

FIG. 13 is an example block diagram illustrating a system utilized to execute the processes described herein, according to an embodiment. The system 1300 includes a processing circuitry 1310 coupled to a memory 1315, a storage 1320, and a network interface 1330. In an embodiment, the components of the system 1300 may be communicatively connected via a bus 1340.

The processing circuitry 1310 may be realized as one or more hardware logic components and circuits. For example, and without limitation, illustrative types of hardware logic components that can be used include field programmable gate arrays (FPGAs), application-specific integrated circuits (ASICs), application-specific standard products (ASSPs), system-on-a-chip systems (SOCs), general-purpose microprocessors, microcontrollers, digital signal processors (DSPs), and the like, or any other hardware logic components that can perform calculations or other manipulations of information.

The memory 1315 may be volatile (e.g., RAM, etc.), non-volatile (e.g., ROM, flash memory, etc.), or a combination thereof. In one configuration, computer readable instructions to implement one or more embodiments disclosed herein may be stored in the storage 1320.

In another embodiment, the memory 1315 is configured to store software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the one or more processors, cause the processing circuitry 1310 to perform the various processes described herein.

The storage 1320 may be magnetic storage, optical storage, and the like, and may be realized, for example, as flash memory or other memory technology, CD-ROM, Digital Versatile Disks (DVDs), or any other medium which can be used to store the desired information.

The network interface 1330 allows the system 1300 to communicate with a data source.

It should be understood that the embodiments described herein are not limited to the specific architecture illustrated in FIG. 13, and other architectures may be equally used without departing from the scope of the disclosed embodiments.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform, such as an additional data storage unit and a printing unit. Furthermore, a non-transitory computer readable medium is any computer readable medium except for a transitory propagating signal.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; A and B in combination; B and C in combination; A and C in combination; or A, B, and C in combination.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A method for gathering and analyzing electronic records comprising:
collecting user value statements;
determining personal product differentiation (PPD) scores based on the user value statements;
collecting experiences related to a user reaction to a product;
analyzing the collected experiences based on the PPDs; and
generating at least a recommendation of a preferred product for the user.

2. The method of claim 1, wherein the user value statements includes patient value statements (PVS), and wherein collecting PVS further comprises:
collecting a patient's demographic information;
identifying at least one disease area;
collecting prioritized symptoms; and
collecting prioritized preferences.

3. The method of claim 2, wherein the collected prioritized preferences include at least one of: routes of administration, unwanted side effects, cost, and lifestyle concerns.

4. The method of claim 1, wherein determining the personal product differentiation (PPD) scores further comprises:
gathering key inputs, wherein key inputs include at least one of: patient medical records, patient healthcare formulary, drug interactions, and product details; and
analyzing how each product best meets the PVS while eliminating adverse drug interactions.

5. The method of claim 1, wherein the collected experiences includes treatment experiences, and wherein collecting treatment experiences further comprises:
gathering product monitoring requirements;
establishing a tailored monitoring plan;
gathering patient treatment experiences based on the tailored monitoring plan; and
updating at least one blinded patient experience database.

6. The method of claim 5, wherein analyzing the collected patient experiences further comprises using artificial intelligence on the collected treatment experiences.

7. The method of claim 6, wherein using artificial intelligence on the collected treatment experiences further comprises:
analyzing features extracted from at least one key database, wherein the least one key database includes any one of: a blinded patient experience database and a blinded patient information database

8. The method of claim 7, further comprising:
using artificial intelligence routines to identify one or more of trends, predictions, learnings, and commonalities.

9. The method of claim 1, wherein the electronic records are human experience records, and wherein the preferred product is a drug in a specific disease area. 10.

10. The method of claim 1, further comprising:
generating a user interface; and
displaying, through the user interface, at least one of: product names, product logos, active ingredients, approval dates, medical history information, administration information, side effect information, and indicator labels.

11. A non-transitory computer readable medium having stored thereon instructions for causing a processing circuitry to execute a process, the process comprising:
collecting user value statements;
determining personal product differentiation (PPD) scores based on the user value statements;
collecting experiences related to a user reaction to a product;
analyzing the collected experiences based on the PPDs; and
generating at least a recommendation of a preferred product for the user.

12. A system for gathering and analyzing human medical treatment experiences, comprising:
a processing circuitry; and
a memory, the memory containing instructions that, when executed by the processing circuitry, configure the system to:
collect user value statements;
determine personal product differentiation (PPD) scores based on the user value statements;
collect experiences related to a user reaction to a product;
analyze the collected experiences based on the PPDs; and
generate at least a recommendation of a preferred product for the user.

13. The system of claim 12, wherein the user value statements includes patient value statements (PVS), and wherein the system is further configured to:
collect a patient's demographic information;
identify at least one disease area;
collect prioritized symptoms; and
collect prioritized preferences.

14. The system of claim 13, wherein the collected prioritized preferences include at least one of: routes of administration, unwanted side effects, cost, and lifestyle concerns.

15. The system of claim 13, wherein the system to is further configured to:
gather key inputs, wherein key inputs include at least one of: patient medical records, patient healthcare formulary, drug interactions, and product details; and
analyze how each product best meets the PVS while eliminating adverse drug interactions.

16. The system of claim 12, wherein the collected experiences includes treatment experiences, and wherein the system is further configured to:
gather product monitoring requirements;
establish a tailored monitoring plan;
gather patient treatment experiences based on the tailored monitoring plan; and
update at least one blinded patient experience database.

17. The system of claim 16, wherein the system is further configured to use artificial intelligence on the collected treatment experiences.

18. The method of claim 17, wherein the system is further configured to:
analyze features extracted from at least one key database, wherein the least one key database includes any one of: a blinded patient experience database and a blinded patient information database

19. The system of claim 18, wherein the system is further configured to:
use artificial intelligence routines to identify one or more of trends, predictions, learnings, and commonalities.

20. The system of claim 12, wherein the electronic records are human experience records, and wherein the preferred product is a drug in a specific disease area.

21. The method of claim 12, wherein the system is further configured to:
generate a user interface; and
display, through the user interface, at least one of: product names, product logos, active ingredients, approval dates, medical history information, administration information, side effect information, and indicator labels.
